(19) United States
(12) Patent Application Publication
(10) Pub. No.: US 2024/0359056 A1
(43) Pub. Date: Oct. 31, 2024

(54) PROTECTIVE PACING ENGINE AND MEASUREMENT PLATFORM

(71) Applicant: Stryd, Inc., Boulder, CO (US)

(72) Inventors: Kristine L. Snyder, Boulder, CO (US); Wyatt Mohrman, Boulder, CO (US); James Williamson, Broomfield, CO (US); Kun Li, Broomfield, CO (US)

(73) Assignee: Stryd, Inc., Boulder, CO (US)

(21) Appl. No.: 18/646,421

(22) Filed: Apr. 25, 2024

Related U.S. Application Data
(60) Provisional application No. 63/498,423, filed on Apr. 26, 2023.

(51) Int. Cl. A63B 24/00

(52) U.S. Cl. CPC A63B 24/0062; A63B 2024/0065; A63B 2220/40; A63B 2220/803

(57) ABSTRACT

The technology relates to a measurement system that is retained by an athlete during an athletic activity. In an example, the measurement system includes a housing that includes: an accelerometer; a gyroscope; at least one processor; and at least one memory, the at least one memory storing instructions that, when executed by the at least one processor cause the system to perform operations. In an example, the operations include receiving accelerometer data and gyroscope data generated by the accelerometer and the gyroscope during a ground-contact portion of a stride; generating one or more impact metrics based on at least one of the accelerometer data or the gyroscope data; generating a real-time pacing value based on the generated one or more impact metrics; and causing a physical output based on the generated real-time pacing value.

### CROSS-REFERENCE TO RELATED APPLICATIONS

This application claims the benefit of U.S. Provisional Application No. 63/498,423 filed Apr. 26, 2023, titled “Protective Pacing Engine and Measurement Platform,” which is incorporated herein by reference in its entirety.

### BACKGROUND

As athletes perform physical activities, such as running, the athlete may unintentionally push themselves to levels that can ultimately be detrimental to the performance and potentially lead to injuries of the athlete. It is with respect to these and other considerations that examples of the technology discussed herein have been made. In addition, although relatively specific problems have been discussed, it should be understood that the examples should not be limited to solving the specific problems identified in the background.

### SUMMARY

This summary is provided to introduce a selection of concepts in a simplified form that are further described below in the Detailed Description section. This summary is not intended to identify key features or essential features of the claimed subject matter, nor is it intended as an aid in determining the scope of the claimed subject matter.

In an aspect, the technology relates to a measurement system that is retained by an athlete during an athletic activity. The measurement system includes a housing that includes: an accelerometer; a gyroscope; at least one processor; and at least one memory, the at least one memory storing instructions that, when executed by the at least one processor cause the system to perform operations. The operations include receiving accelerometer data and gyroscope data generated by the accelerometer and the gyroscope during a ground-contact portion of a stride; generating one or more impact metrics based on at least one of the accelerometer data or the gyroscope data; generating a real-time pacing value based on the generated one or more impact metrics; and causing a physical output based on the generated real-time pacing value.

In an example, the operations further include determining an initial impact time for the stride; integrating a vertical acceleration component of the acceleration data over an initial impact period; and wherein generating the one or more impact metrics is based on the integration of the vertical acceleration component. In another example, the operations further include determining speed and incline at an impact with the ground during the stride; and wherein generating the one or more impact metrics is based on speed and incline. In still another example, causing the physical output includes generating at least one of an audio output, a visual output, or a haptic output. In yet another example, the operations further include based on the accelerometer data and gyroscope data, generating a modeled force curve for the ground-contact portion of the stride; and based on the modeled force curve, determining an impact loading rate for the stride, wherein the impact loading rate is one of the one or more impact metrics. In a further example, generating the impact loading rate includes determining a slope of the modeled force curve. In a still further example, the slope of the modeled force curve occurs in a section of the force curve between a starting point of the force curve and a first peak of the force curve.

In another example, the operations further include calculating the impact loading rates for a plurality of strides during the athletic activity; and generating a lower body stress value by accumulating the calculated impact loading rates. In a further example, the athletic activity is a first athletic activity and the operations further include, during a second athletic activity generating a second pacing value based on the lower body stress value. In yet another example, the housing is a foot pod that is configured to attach to a shoe during the athletic activity.

In another aspect, the technology relates to a measurement system that is retained by an athlete during an athletic activity. The measurement system includes one or more housings that house: an inertial measurement unit including an accelerometer and a gyroscope; a global positioning sensor; at least one environmental sensor; at least one processor; and at least one memory, the at least one memory storing instructions that, when executed by the at least one processor cause the system to perform operations. The operations include based on accelerometer and gyroscope data generated by the accelerometer and the gyroscope, generating one or more real-time mechanical inputs; based on environmental data generated by the at least one environmental sensor, generating one or more real-time environmental inputs; accessing at least one of a metabolic static input or an equipment static input; based on the at least one of a metabolic static input or an equipment static input, adjusting an initialization pacing target to form an adjusted initialization pacing target; based on the one or more real-time environmental inputs and the one or more real-time mechanical inputs, generating a real-time pacing value; and causing a physical output based on the generated real-time pacing value.

In an example, the initialization pacing target is a target range having a maximum pacing threshold and a minimum pacing threshold; the adjusted initialization pacing target includes an adjusted maximum pacing threshold and an adjusted minimum pacing threshold; and the real-time pacing value is between the adjusted maximum pacing threshold and the adjusted minimum pacing threshold. In another example, the metabolic static input includes at least one of recovery, sleep, or nutrition. In yet another example, the equipment input includes at least one of apparel data or shoe data. In still another example, the one or more real-time mechanical inputs include at least one of impact loading rate, negative mechanical power, or positive mechanical power. In still yet another example, the one or more real-time environmental inputs include ground surface data. In a further example, the one or more real-time environmental inputs include incline data, wherein the incline data is generated from querying an aggregate trail map based with global navigation satellite system (GNSS) data generated from the global positioning sensor during the athletic activity.

In another aspect, the technology relates to a measurement system that is retained by an athlete during an athletic activity. The measurement system includes one or more housings that house: an inertial measurement unit including an accelerometer and a gyroscope; a barometer; a temperature sensor; at least one processor; and at least one memory, the at least one memory storing instructions that, when executed by the at least one processor cause the system to perform operations. The operations include determining an altitude based on at least one of measurements from the barometer or position data received from a global positioning sensor; measuring, by the temperature sensor, a temperature of an environment around the measurement system; generating a real-time pacing value based on the altitude and temperature; and causing a physical output based on the generated real-time pacing value.

In an example, an increase in altitude causes a reduction in the real-time pacing value. In another example, an increase in temperature causes a reduction in the real-time pacing value. In yet another example, the system further includes the global positioning sensor. In still another example, the system further includes a hygrometer, and the operations further comprise measuring, by the hygrometer, a humidity level of an environment around the measurement system. In a further example, generating the real-time pacing value is further based on the humidity level. In a still further example, an increase in humidity causes a reduction in the real-time pacing value.

In another aspect, the technology relates to a computer-implemented method for generating an aggregate trail map having high-resolution incline data. The method includes acquiring global navigation satellite system (GNSS) data from multiple measurement platforms over multiple runs along a trail, wherein each measurement platform includes at least a global positioning sensor and a barometer; acquiring pressure data from the measurement platforms over the multiple runs; correlating the GNSS data with the pressure data; combining the correlated GNSS data and the pressure data to form an aggregate data set; from the aggregate data set, generate incline data for global positions of the trail; and generating the aggregate trail map with the incline data and the GNSS data from the aggregate data set; and storing the aggregate trail map.

In an example, the method further includes during a run on the trail by an athlete retaining a measurement platform, receiving real-time GNSS data by the measurement platform, the GNSS data corresponding to a particular global position on the trail; and querying, by the measurement platform, the aggregate trail map with the received GNSS data to retrieve corresponding incline data to the particular global position on the trail. In another example, the method further includes generating a real-time pacing value based on the corresponding incline data; and causing a physical output based on the generated real-time pacing value.

The details of one or more aspects are set forth in the accompanying drawings and description below. Other features and advantages will be apparent from a reading of the following detailed description and a review of the associated drawings. It is to be understood that the following detailed description is explanatory only and is not restrictive of the invention as claimed.

### BRIEF DESCRIPTION OF THE DRAWINGS

The accompanying drawings, which are incorporated in and constitute a part of this disclosure, illustrate various aspects of the present invention. In the drawings:

FIG. 1A depicts a perspective view of a measurement platform in the form of a foot pod attached to a shoe.

FIG. 1B depicts an exploded view of the foot pod of FIG. 1A.

FIG. 2 depicts an example measurement platform.

FIG. 3 depicts example positions for components of an example measurement platform.

FIG. 4A depicts an example pacing system.

FIG. 4B depicts an example method for adjusting pacing outputs.

FIGS. 5A-C depict example plots of force curves.

FIG. 5D depicts an example method for generating the modeled force curves.

FIG. 6 depicts an example annotated force curve.

FIG. 7A depicts an example force plot.

FIG. 7B depicts an example energy plot based on the force plot of FIG. 7A.

FIG. 8 depicts an example energy return plot.

FIG. 9 depicts an example energy return plot for a sample of different shoes.

FIG. 10 depicts an example topographic aggregate trail map.

FIG. 11 depicts an example method for generating and using an aggregate trail map.

### DETAILED DESCRIPTION

The following detailed description refers to the accompanying drawings. Wherever possible, the same reference numbers are used in the drawing and the following description to refer to the same or similar elements. While aspects of the invention may be described, modifications, adaptations, and other implementations are possible. For example, substitutions, additions, or modifications may be made to the elements illustrated in the drawings, and the methods described herein may be modified by substituting, reordering, removing, or adding stages or operations to the disclosed methods. Accordingly, the following detailed description does not limit the invention, but instead, the proper scope of the invention is defined by the appended claims. Examples may take the form of a hardware implementation, or an entirely software implementation, or an implementation combining software and hardware aspects. The following detailed description is, therefore, not to be taken in a limiting sense.

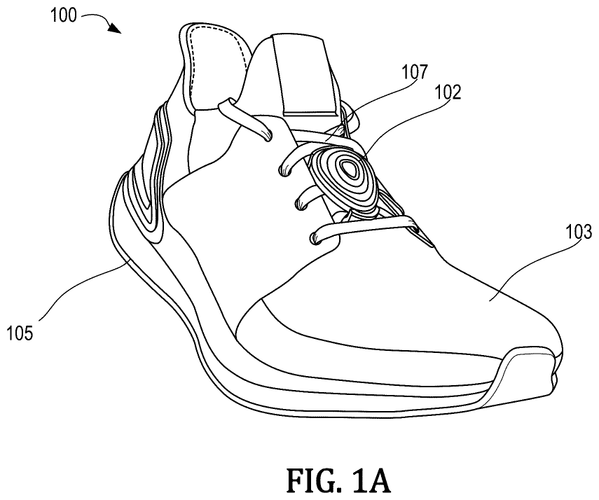

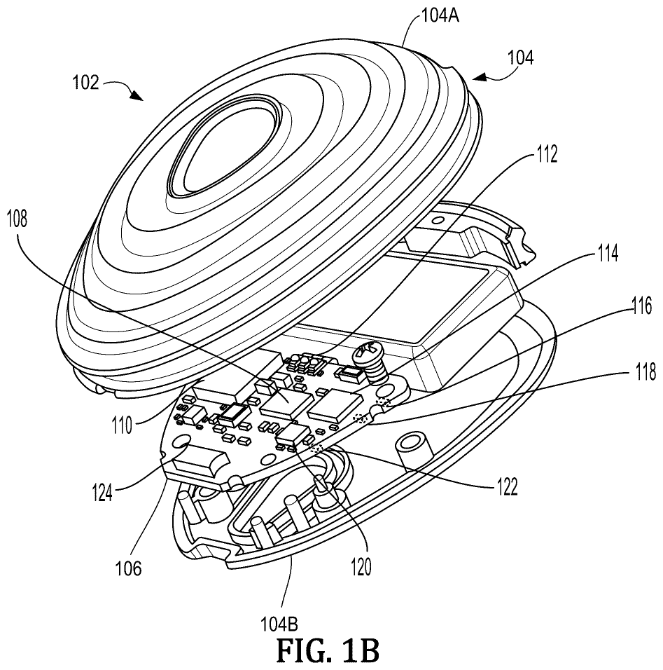

FIG. 1A depicts a perspective view of a measurement platform 100 in the form of a foot pod 102 attached to a shoe 103. As discussed further below, the foot pod 102 may house measurement components that allow for protective pacing feedback to be generated for the athlete. By attaching the foot pod 102 and the sensors therein to the shoe 103 of the athlete, higher integrity data may be obtained about the movement of the foot of the athlete.

In the example depicted, the foot pod 102 attaches to the exterior of the shoe 103. More specifically, the foot pod 102 attached to the laces 107 of the shoe 103. The foot pod 102 may include a detachable clip that can be securely clipped to the laces. In other examples, the foot pod 102 may be attached to the exterior of the shoe 103 in other manners or be attached to different portions of the shoe 103. For instance, the foot pod 102 (or the components thereof) may be integrated into the shoe 103. As an example, the components of the foot pod 102 may be integrated into the sole 105 of the shoe 103, among other locations. While the foot pod 102 is shown as connected to the shoe 103, in other examples the foot pod 102 may be a worn pod that can be clipped or connected to other parts of the athlete or the athlete's clothing (e.g., shorts or shirt). While only a single shoe 103 and foot pod 102 are shown in FIG. 1A, in some examples, a foot pod 102 may be attached to each shoe 103 of the athlete and data from both foot pods 102 may be used in the technology discussed herein.

FIG. 1B depicts an exploded view of the foot pod 102 of FIG. 1A. The example foot pod 102 includes a housing 104 composed of a top shell 104A and a bottom shell 104B that connects to the top shell 104A to form the housing 104. The housing 104 defines an inner cavity that houses a plurality of computing and measurement components of the measurement platform 100. The computing and measurement components may be included on a circuit board 106 within the housing 104. For instance, the measurement components may include at least a processor 108, memory 110, multiple accelerometers 112, 114, 116, a gyroscope 118, a magnetometer 120, environmental sensors 122, and a radio-frequency (RF) antenna 124. The environmental sensors 122 may include sensors such as temperature, pressure, and/or humidity sensors.

The multiple accelerometers 112-116 may each be three-axis accelerometers that generate multi-axis motion data. The first accelerometer 112 may provide a wake function that creates a wake signal upon movement to activate the system. The second accelerometer 114 may provide a high-dynamic range signal, and the third accelerometer 114 may provide a low-dynamic range signal. In other examples, only a single accelerometer may be used. The gyroscope 118 may be used to generate orientation data. The orientation data in combination with the motion data from the accelerometer(s) may be used to generate additional metrics as discussed herein. The magnetometer 120 may also generate orientation data, and in some examples, either the gyroscope 118 or the magnetometer 120 may be omitted. The combination of an accelerometer with a gyroscope and/or a magnetometer may be referred to herein as an inertial measurement unit (IMU) that generates or outputs multi-axis motion data.

The processor 108 may be a microprocessor, such as a central processing unit (CPU). In other examples, the processor 108 may be a field-programmable gate array (FPGA) or an applications-specific integrated circuit (ASIC). The memory 110 may include volatile storage (e.g., random access memory), non-volatile storage (e.g., read-only memory), flash memory, or any combination of such memories.

The radio-frequency (RF) antenna 124 may be used to communicate data from the foot pod 102 and/or received data at the foot pod 102. For instance, the multi-axis motion data may be communicated from the foot pod 102 to a secondary device, such as a smartphone or watch that is carried or worn by the athlete during the athletic activity.

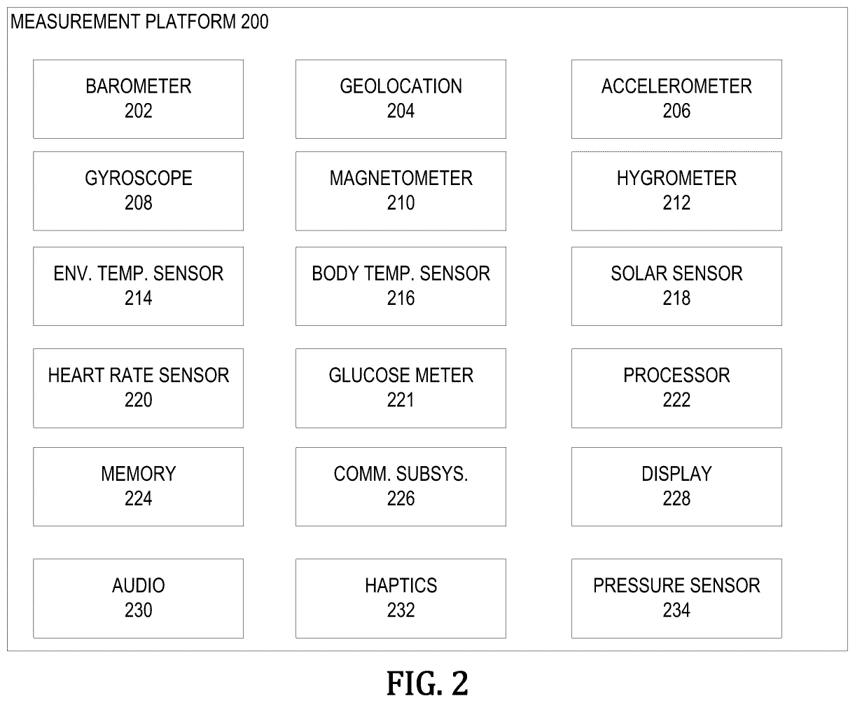

FIG. 2 depicts an example measurement platform 200. The example measurement platform 200 may be similar to the measurement platform 100 discussed above. The example measurement platform includes a barometer 202, a geolocation receiver 204, an accelerometer 206, a gyroscope 208, a magnetometer 210, a hygrometer 212, an environmental temperature sensor 214, a body temperature sensor 216, a solar sensor 218, a heart rate sensor 220, a glucose meter 221, a processor 222, memory 224, a communications subsystem 226, a display 228, audio output 230, haptics 232, and/or a pressure 234. In some examples, not all of the computing and measurement components of the measurement platform 200 may be included, and in some examples, additional computing and measurement components are included in the measurement platform 200.

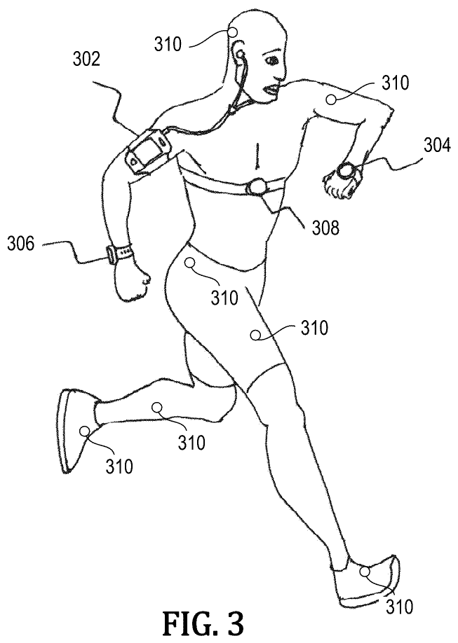

In some examples, the components of the measurement platform 200 may be distributed across multiple distributed devices of the athlete. For instance, FIG. 3 depicts example positions and devices for components of an example measurement platform, such as measurement platform 200. For example, the athlete may be wearing one or more wrist-based devices, such as a sports watch 306 and/or a smartwatch 304. The athlete may also or alternatively be wearing a chest-strap device 308 and/or have a smartphone 302 that is attached, held, or otherwise retained by the athlete.

The various computing and measurement components of the measurement platform 200 may be distributed in different manners across the devices of the athlete, such as any worn device of the athlete (e.g., foot pod 102, watches 304, 306, chest strap 308) and/or retained by the athlete while performing the athletic activity (e.g., smart phone 302). For instance, the feedback components (e.g., display 228, audio 230, and/or haptics 232) may be incorporated into a smartphone 302 and/or a watch 304, 306. The remaining components may then be housed in a foot pod 102, another wearable/clipped pod, a chest strap device 308, and/or integrated into the shoe itself or a portion of shoe, such as an insole. Each of the devices (e.g., foot pod 102, watches 304, 306, chest strap 308, smart phone 302) may have its own housing that houses one or more of the components of the measurement platform. In some examples, all the components of the measurement platform 200 are incorporated into a single device, such as watch 304, 306 or pod 102. The measurement platform 200 may also be distributed across different housings 310 that are retained by or attached to the athlete (via straps or clothing). For instance, the housings may be on the front, back, or side of the torso, on the front, back, or side of the waist, embedded in clothing, attached to or embedded within one or both shoes, on one or both socks, on the front or back of a headband, on one or both thighs, in one or both arm bands, or on one or both calves. Depending upon the implementation, each location can have one or more of the following advantages: convenience to the user, accuracy of measuring running and/or walking technique and distance, accuracy in measuring the geographic position of the user, improved sensed data quality, improved user comfort, monitoring of one or more limbs of a user; and reduced stress on the sensing device. By way of example, beneficial locations can include within or on a strap attached to the ankle, leg, wrist, waist, or torso. The sensor platform, or portions thereof, can also be placed within or on apparel such as clothing, belts, or shoes. It can also be placed on, under, or to the side of the foot.

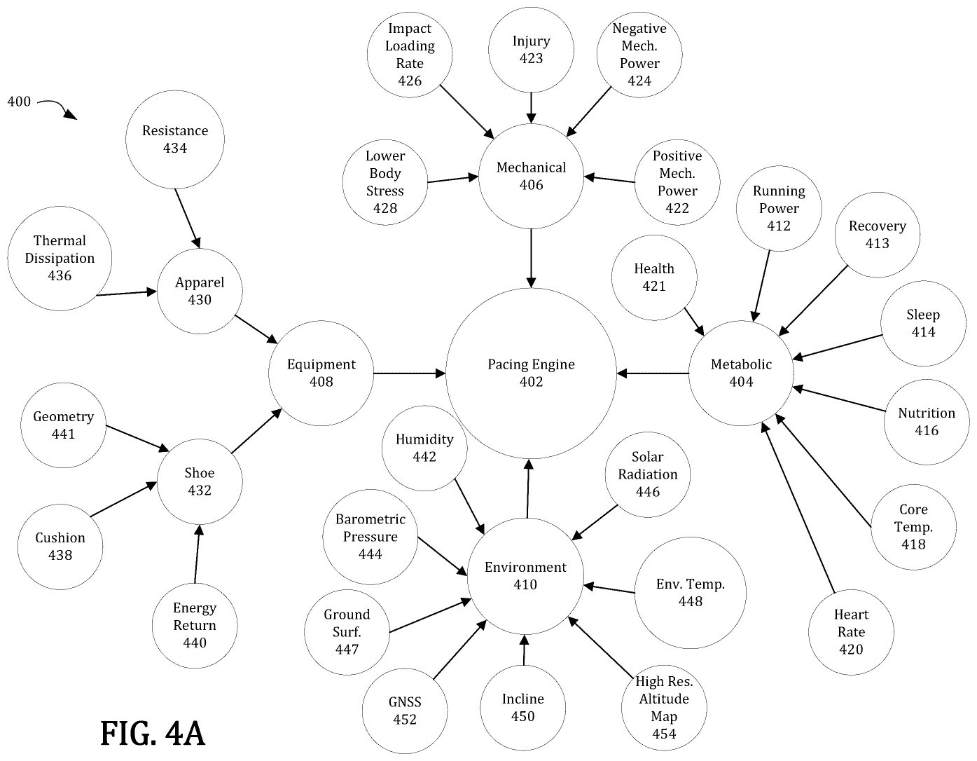

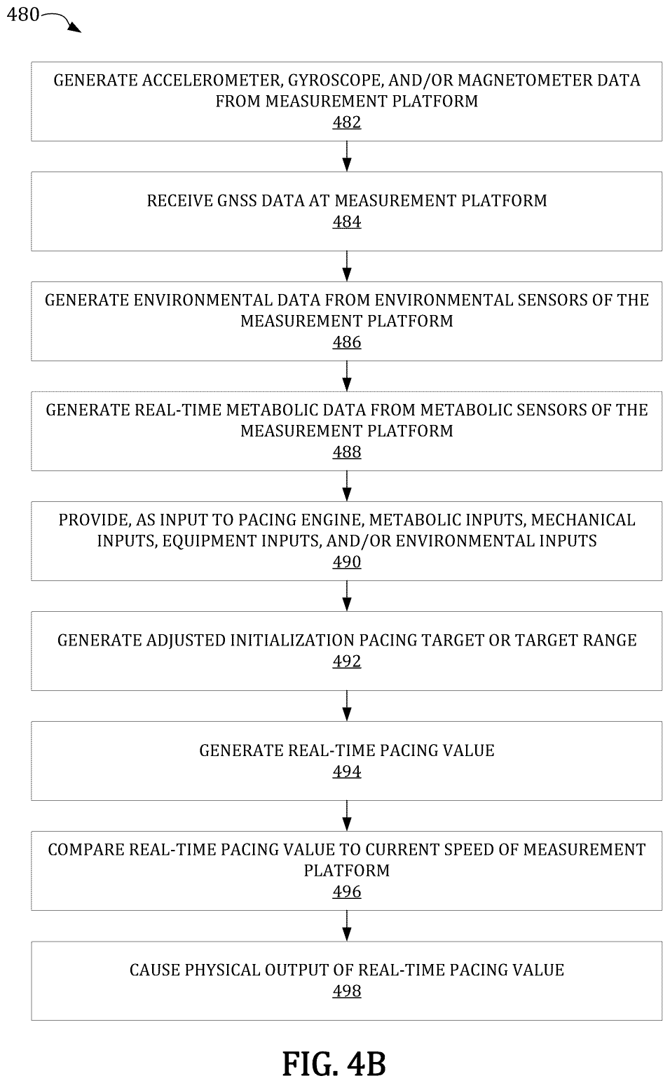

FIG. 4A depicts an example pacing system 400. The example pacing system 400 includes a pacing engine 402 that receives a plurality of inputs that are processed to generate protective pacing feedback or outputs as the athlete is performing the physical activity with a measurement platform. In some examples, the pacing engine 402 represents an algorithm or model that processes the inputs and generates the pacing outputs discussed below. Alternatively, in addition, or in combination, the pacing engine 402 may utilize a trained machine learning (ML) model to process the inputs and generate the pacing inputs discussed herein. The ML model may be trained using supervised (or unsupervised) training methods on data sets from a plurality of runners captured over time that have been tagged based on positive and negative pacing outcomes and/or outcomes that led to injury or otherwise degraded performance.

The inputs that are received by the pacing engine 402 may be real-time inputs (e.g., inputs that are generated and updated throughout the physical activity) or static inputs (e.g., inputs that do not change during the physical activity). As example inputs, the pacing engine 402 may receive metabolic inputs 404, mechanical inputs 406, equipment inputs 408, and environmental inputs 410.

The pacing engine 402 may have an initialization pacing target or target range having a maximum pacing threshold and a minimum pacing threshold. The initialization pacing target or target range may be based on the type of activity for which the athlete is participating. For instance, pacing may be different for a marathon than for a high intensity interval training. The initialization pacing target or target range is also then adjusted based on the particular athlete, the goals for that athlete, and/or the training targets and plan for that athlete that are generated or programmed into the measurement platform. The initialization pacing target or target range may be represented as a particular speed for the athlete to run. In other examples, the pacing target may be a particular power output (or power output range) for athlete during the run or another target metric based on the pacing-engine determinations discussed herein.

The initialization pacing target or target range may then also be adjusted based on the static inputs of the metabolic inputs 404, mechanical inputs 406, equipment inputs 408, and environmental inputs 410. The effects of the different inputs on the adjustment initialization pacing target or target range are discussed below. During the athletic activity, the pacing engine 402 generates a real-time pacing output that is based on the real-time data or inputs of the metabolic inputs 404, mechanical inputs 406, equipment inputs 408, and environmental inputs 410. For instance, based on real-time changes of the athlete and/or the user, the real-time pacing output may be adjusted to provide a pacing output that may help prevent injury while also helping the athlete continue to improve performance and/or increase efficiency. The effects of the different inputs on the real-time pacing output are discussed in further detail below.

The metabolic inputs 404 may include running power 412, a recovery input 413, a sleep input 414, nutrition input 416, health input 421, running power 412, a core temperature input 418, and/or a heart rate input 420, among others. The sleep input 414, recovery input 413, and nutrition input 416 may be static inputs that are received via user input into the measurement platform.

In some examples, the nutrition input 416 may be provided by the glucose meter. Depending on the type of glucose meter utilized, the nutrition input 416 from the glucose meter may be a static input captured prior to the athletic activity or a real-time input that is measured during the athletic activity.

In some examples, the sleep input 414 may be generated from a sleep tracking application that relies on measurements from the accelerometer. The recovery input 413 represents recent workout and recovery time or activities for the athlete. For instance, the recovery input 413 may include the frequency and intensity of prior athletic activities by the athlete over the prior week or set number of days. The recovery input 413 may be based on activity tracking by the measurement platform or through input from the athlete regarding the recent activity of the athlete. The health input 421 may be received as input from the athlete indicating whether the athlete is healthy or sick. For instance, when the athlete is sick, the athlete may provide that input into the measurement platform.

The running power 412, core temperature input 418, and heart rate input 420 in contrast may be real-time inputs that are measured by the heart rate sensor and/or the temperature sensor of the measurement platform during the athletic activity. The running power 412 may be generated as discussed in U.S. Patent Publication 2017/0189752, filed on Mar. 20, 2017, and U.S. Patent Publication 2020/0405231, filed on Jun. 26, 2020, which are both incorporated herein by reference in their entireties. To the extent the present application conflicts with any portion of those applications incorporated by reference, the disclosure in the present application shall control.

The core temperature input 418 may be generated from the body temperature sensor of the measurement platform. The heart rate input 420 may be generated from the heart rate sensor of the measurement platform.

The pacing engine 402 may use the sleep input 414, recovery input 413, health input 421, and nutrition input 416 to adjust the initialization pacing target or target range. For example, if the sleep input 414 indicates that the athlete slept poorly in the recent past (e.g., the night before), the initialization pacing target or target range may be reduced. In contrast, if the sleep input 414 indicates a good sleep history, the initialization pacing target or target range may be maintained or increased. The recovery input 413 and the nutrition input 416 may be similarly used. For example, if the recovery input 413 indicates a high frequency of recent activity with limited recovery time, the initialization pacing target or target range may be reduced. If the recovery input 413 indicates large amounts of recovery time, the initialization pacing target or target range may be maintained or increased. If the nutrition input 416 indicates poor nutrition in the recent past (e.g., same day, prior day, prior week), the initialization pacing target or target range may be reduced. If the nutrition input 416 indicates good nutrition, the initialization pacing target or target range may be maintained or increased. If the health input 421 indicates that the athlete is sick, the initialization pacing target or target range may be reduced.

During the athletic activity, while the measurement platform is worn or carried by the athlete, the pacing engine 402 may use the running power 412, core temperature input 418, and heart rate input 420, to generate a real-time pacing output from the adjusted initialization pacing target or target range. For example, a running power 412 that is higher than a target running power for the athlete may cause the real-time pacing output to be reduced. A running power 412 that is lower than a target running power for the athlete may cause the real-time pacing output to be increased. A core temperature input 418 that is outside of a range of a normal temperature for the human body (e.g., a set threshold outside of 98.6 degrees Fahrenheit) may cause the real-time pacing output to be reduced as there may be a potential health issue with the athlete. Similarly, if the heart rate input 420 indicates a high heart rate above a target heart rate for the athlete (e.g., based on the athlete's size, age, and/or athletic history), the real-time pacing output may be reduced to help protect the athlete from injury. Once the heart rate input 420 returns to a normal range, the real-time pacing output may be maintained or increased.

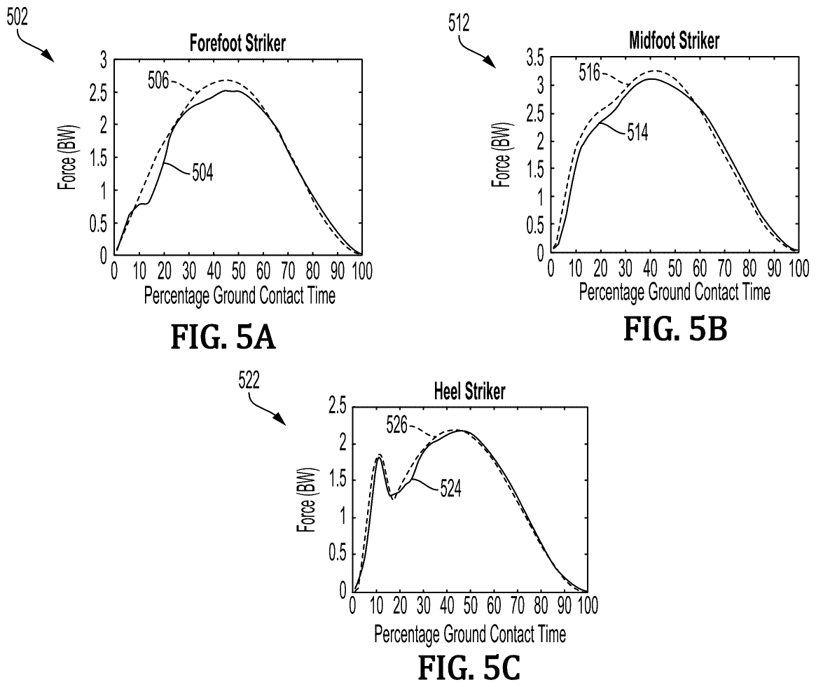

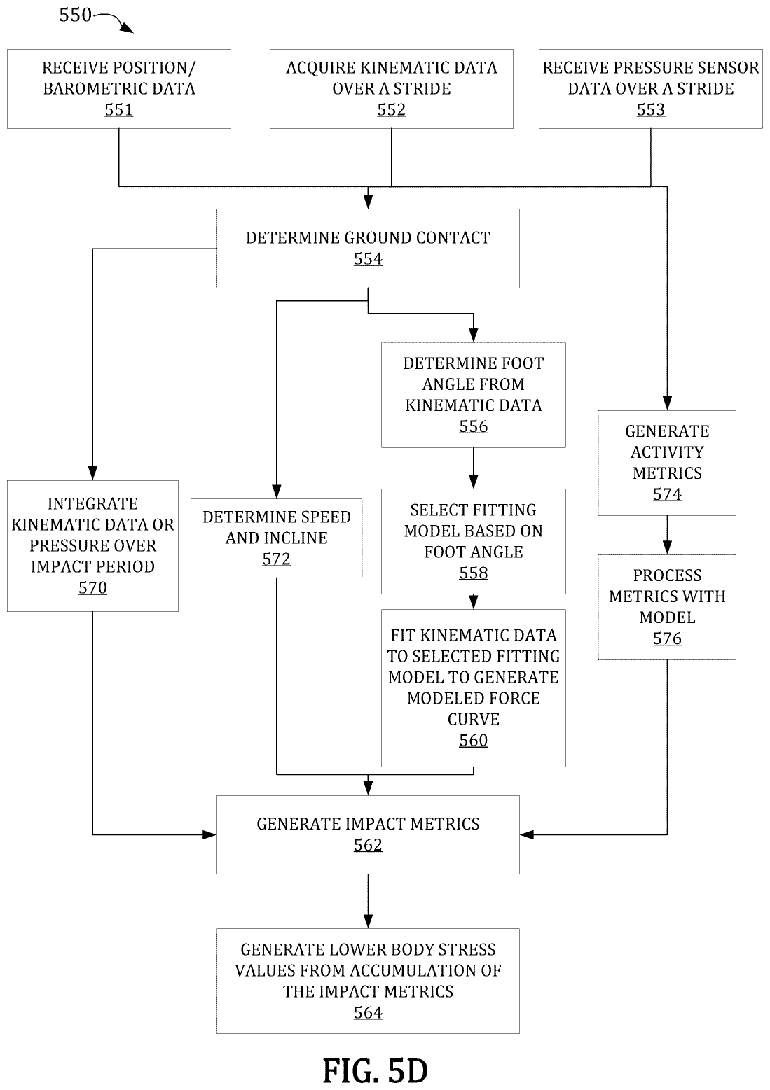

The mechanical inputs 406 may include a positive mechanical power input 422, a negative mechanical power input 424, an impact loading rate 426, and a lower body stress input 428, and an injury input 423 among others. The positive mechanical power input 422, the negative mechanical power input 424, and the impact loading rate 426 may be real-time inputs that are generated during the athletic activity. For instance, the impact loading rate 426 may be generated for each stride. The impact loading rate 426 may be the initial rate of increase in vertical force as the athlete contacts the ground with their foot and may be represented in body weight per second. Higher values of impact loading rate 426 mean that force is being applied at a faster rate. Lower values of the impact loading rate 426 mean that force is being applied at a slowerrate. The impact loading rate may be considered an overall metric of how much load is being applied to the lower body over time. In general, the impact loading rate increases when running downhill, decreases when running uphill, and increases with running speed or running intensity. Examples of generating positive mechanical power input 422, the negative mechanical power input 424, and the impact loading rate 426 are described in further detail below with respect to at least FIGS. 5-7.

The lower body stress input 428 may be a static input that is determined or generated prior to the athletic activity and may not change during the athletic activity itself. For instance, the lower body stress input 428 may represent the mechanical load experienced between the runner's center of mass and the ground that they accumulate during their day-to-day activities. The lower body stress input 428 may be based on the impact loading rate 426 that has been accumulated over a set period of time.

The initialization pacing target or target range may be adjusted based on the lower body stress input 428 that has accumulated from the athlete over a set time frame (e.g., a week, two weeks, etc.). For example, if the lower body stress input 428 is higher than average for the athlete or above a threshold, the pacing engine 402 may reduce the initialization pacing target or target range. In contrast, if the lower body stress input 428 is below average for the athlete or below a threshold, the initialization pacing target or target range may be maintained or increased.

The injury input 423 may also be a static input that is determined or generated prior to the athletic activity and may not change during the athletic activity itself. The injury input 423 may be an input that is received from the athlete into the measurement platform. In other examples, the injury input 423 may be detected from the measurement platform itself. For instance, over multiple runs of athletic activities by same athlete, a baseline for that athlete's form or measured characteristics may be established. When a divergence from the baseline is detected (e.g., a measured characteristic outside of a threshold), an injury of the athlete may be determined or predicted. The predicted existence of an injury may be used as the injury input 423. In some examples, when an injury is predicted, a confirmation or other user interface element may be provided to the athlete to have the athlete confirm the presence of the injury.

The initialization pacing target or target range may be adjusted based on the injury input 423. For example, if the injury input indicates that the athlete is currently injured, the pacing engine 402 may reduce the initialization pacing target or target range. In contrast, if the injury input 423 does not indicate the presence of an injury, the initialization pacing target or target range may be maintained.

The pacing engine 402 may then generate a real-time pacing output based further on the impact loading rate 426, the negative mechanical power input 424, and/or the positive mechanical power input 422. For example, when the impact loading rate 426 is higher than average for the athlete or above a threshold, the real-time pacing output may be reduced to a slower pace. When the impact loading rate 426 is lower than average for the athlete or below a threshold, the real-time pacing output may be increased to a faster pace.

The negative mechanical power input 424 represents the amount of power that is lost due to the interaction with the athlete's foot and the ground. The positive mechanical power input 422 represents the amount of power that is put back into the stride. The positive mechanical power input 422 is a combination of the recycled energy from the running stride (such as the spring force from the leg, foot, and shoe) as well as the metabolic energy expended by the athlete. In order for the athlete to maintain a particular pace on a flat surface, the positive mechanical power input 422 must be the same or greater than the negative mechanical power input 424. To accelerate to a higher pace, the positive mechanical power input 422 is greater than the negative mechanical power input 424. While the net difference between the positive mechanical power input 422 and the negative mechanical power input 424 may be about zero when an athlete is maintaining the pace, the magnitude of each of the positive mechanical power input 422 and the negative mechanical power input 424 may be indicative the athlete's form and/or efficiency. In addition, a high negative mechanical power input 424 may be indicative of a higher probability for injury. As such, when the negative mechanical power input 424 is higher than an average or threshold, the real-time pacing output may be reduced. When the negative mechanical power input 424 is lower than an average or threshold, the real-time pacing output may be increased.

The equipment inputs 408 may include apparel inputs 430 and/or shoe inputs 432. The shoe inputs 432 may include cushion data 438, energy return data 440, and/or shoe geometry data 441. The apparel inputs 430 may include thermal dissipation data 436 and/or resistance data 434, which may include air resistance and/or resistance to movement (e.g., restrictive clothing).

The apparel inputs 430 may be static inputs that are received via user input prior to the athletic activity. For instance, the athlete may input into the measurement platform the type of apparel that the athlete is wearing during the upcoming athletic activity. A database storing thermal dissipation data 436 and/or resistance data 434 may be queried based on the type of apparel entered by the athlete.

The shoe inputs 432 may also be static inputs that are received via user input prior to the athletic activity. For example, the athlete may input into the measurement platform the type of shoe that the athlete is wearing during the upcoming athletic activity. A database storing cushion data 438 and/or energy return data 440 may be queried based on the type of shoe entered by the athlete. The shoe geometry data 441 may also be used to query or generate the cushion data 438 and/or energy return data 440. The shoe geometry data 441 may include heights, lengths, and/or widths or the shoe or portions thereof, such as a drop height of the heel of the shoe. In some examples, the shoe geometry data 441 may be accessed from an input of a type of shoe (e.g., brand and model) of the shoe.

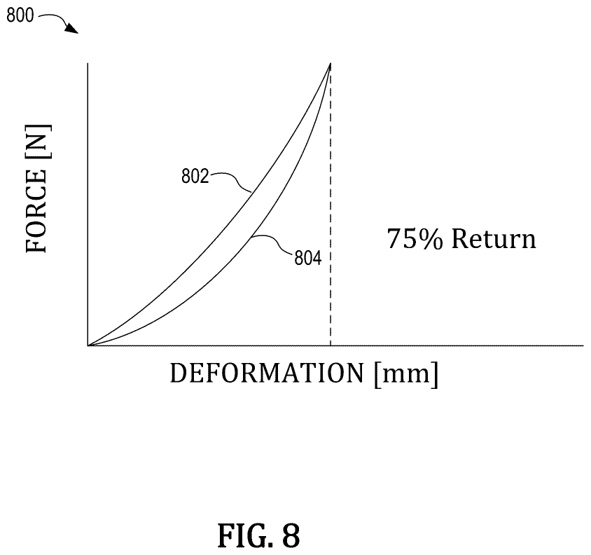

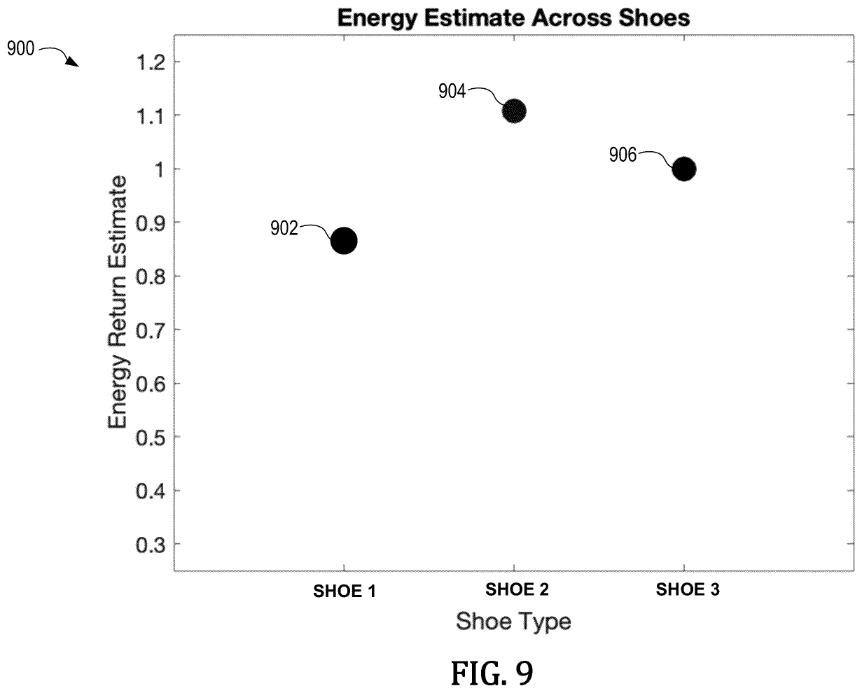

The energy return data 440 and/or the cushion data 438 may also be determined or generated from measurements of the measurement platform. Examples of determining such energy return data 440 and/or the cushion data 438 from the measurement platform is discussed in further detail below with at least reference to FIGS. 8-9.

The pacing engine 402 may use the equipment inputs 408 to adjust the initialization pacing target or target range. For instance, for shoe inputs 432 that indicate more cushion or higher energy return, the initialization pacing target or target range may be increased. Similarly, for apparel inputs 430 that indicate less thermal dissipation and/or higher resistance may reduce the initialization pacing target or target range.

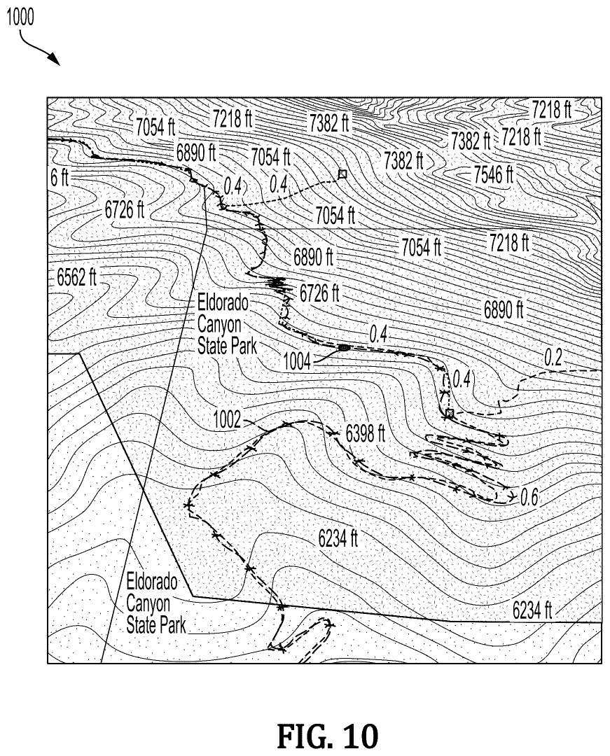

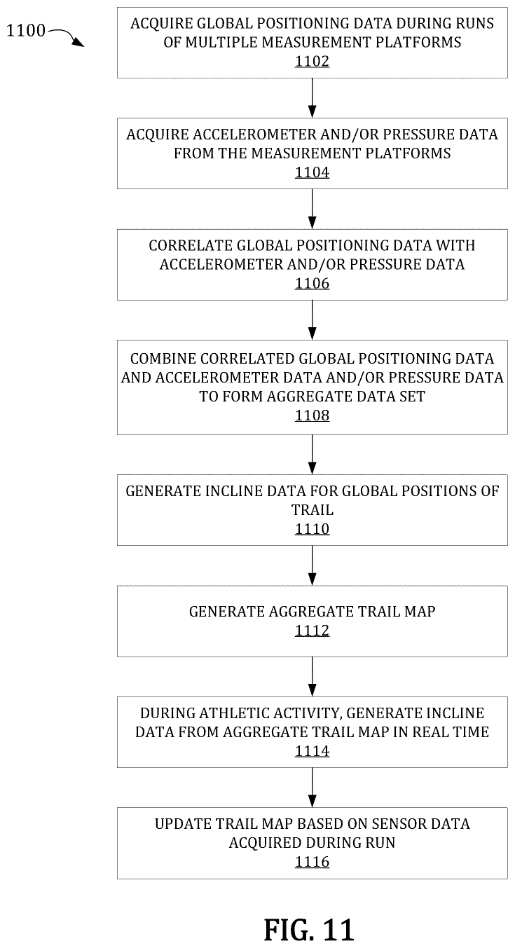

The environmental inputs 410 may include humidity 442, barometric pressure 444, solar radiation 446, ground surface data 447, environmental temperature 448, and incline data 450. The humidity 442 may be generated from the hygrometer of the measurement platform, the barometric pressure 444 may be generated from the pressure sensor of the measurement platform, the solar radiation 446 may be generated from the solar sensor of the measurement platform, and the environmental temperature 448 may be generated from the environmental temperature sensor of the measurement platform. In other examples, the humidity 442, barometric pressure 444, solar radiation 446, ground surface data 447, and/or the environmental temperature 448 may be obtained during the activity from external databases or sources (e.g., current weather sources) via queries or feeds that may be handled by a smart phone or smart watch that is connected to or incorporated into the sensor platform. The incline data 450 may be based on measurements from an accelerometer, pressure sensor, and/or from other sources, such as global navigation satellite system (GNSS) data 452 and/or a high-resolution altitude map 454. The GNSS data may be acquired from the geolocation system or receiver of the measurements platform. Examples of generating and using an aggregate high-resolution trail map to identify incline data is discussed in further detail below with respect to FIGS. 10-11.

The ground surface data 447 may be generated from accelerometer data produced by the measurement platform. For instance, by analyzing the impact forces from the accelerometer of the shoe/foot when it contacts the ground, a type of ground surface may be determined—such as a hard surface (e.g., trail, road), a medium surface (e.g., track), or a soft surface (e.g., sand, snow). As an example, during the push off phase of the stride, ground response forces may be detected from the measurement platform, which may indicate how the ground is responding to the forces. As a more specific example, with the shoe geometry of the shoe known, such as the drop height of the heel, a model for how the shoe or center of pressure measured by the measurement platform moves or rolls through a perfectly rigid surface may be generated or accessed. Then in real-time, the center of pressure may be tracked throughout a stride and compared to the model for the perfectly rigid surface. Deviations from the rigid model indicate a different type of surface. Based on the amount and type of deviation, the type of ground surface may be classified.

In other examples, the ground surface data 447 may be generated based on the GNSS data. For example, a type of trail may be determined based on the location of the measurement platform. Ground surface data for the trail (or portions of the trail) may be stored in a database and accessed based on the GNSS data.

The pacing engine 402 may adjust the real-time pacing output based on the environmental inputs 410. For instance, the humidity 442 and/or the barometric pressure 444 may be used to determine air resistance due to wind. Higher air resistance (e.g., headwind) may cause the real-time pacing output to be decreased. In addition, higher altitude may indicate that there is less oxygen available in the air for consumption. As a result, the real-time pacing output may be reduced at higher altitudes. Higher humidity may also cause the real-time pacing output to be reduced. The effect of humidity on the real-time pacing output may not be linear or may be based on additional factors. For example, when the environmental temperature is high, a small increase in humidity may cause a larger reduction the real-time pacing output than when the environmental temperature is low.

The environmental temperature 448 and/or solar radiation 446 may also be used to modify the real-time pacing output. For example, for temperatures that are above a temperature threshold or solar radiation that is above a solar-radiation threshold, the real-time pacing output may be reduced.

The pacing engine 402 may adjust the real-time pacing output based on the ground surface data 447. For example, where the ground surface is a type of surface that is more difficult to maintain pace (e.g., more energy is needed to maintain pace), the real-time pacing output may be reduced. For example, where the ground surface data 447 indicates a soft surface (e.g., sand, snow), the real-time pacing output may be reduced.

The pacing engine 402 may also use the incline data 450 to adjust the real-time pacing output. For example, when the incline data 450 indicates an uphill incline, the real-time pacing output may be reduced. When the incline data 450 indicates a downhill incline, the real-time pacing output may be increased. However, as discussed above, when running downhill, the impact loading rate 426 may also increase. Accordingly, while a runner may be capable of maintaining a higher pace while running downhill, the runner may also be more prone to injury when doing so because the runner's impact loading rate 426 also increases. The present protective pacing engine 402 accounts for both the mechanical load and physical environment around the user to generate in real time (e.g., second by second or in less than 5 second, or second intervals) pacing outputs that promote athlete performance as well as athlete safety. Such technical achievements and improvements have been previously unachievable and were not otherwise possible in combination. Even determining many of the individual inputs, such as mechanical forces received by the body, were not possible without force plates on the ground or specialty external equipment that could not be worn by the athlete. Accordingly, real-time measurements could not previously be made-especially when running in the real world, such as on a mountain trail.

FIG. 4B depicts an example method 480 for adjusting pacing outputs. The method 480 may be executed or otherwise performed by the measurement platforms discussed herein. For instance, the memory of the measurement platform may include instructions that when executed by one or more processors of the measurement platform, cause the measurement platform to perform the operations described herein. The operations depicted in method 480 may also be performed concurrently and/or in a different order than depicted in FIG. 4B.

At operation 482, accelerometer, gyroscope, and/or magnetometer data (e.g., multi-axis motion data) is generated from a measurement platform in real time while the measurement platform is retained by an athlete during an athletic activity (e.g., attached to and/or carried by an athlete). At operation 484, GNSS data is received at the measurement platform from the global positioning sensor. At operation 486, environmental data is generated from the environmental sensors (e.g., humidity, temperature, pressure, and/or solar radiation sensors) of the measurement platform. At operation 488, real-time metabolic data is generated from the metabolic sensors (e.g., heart rate and body temperature sensors) of the measurement platform.

At operation 490, metabolic inputs, mechanical inputs, and/or environmental inputs are generated and provided as input to the pacing engine of the measurement platform. The inputs may include static inputs discussed above that are determined or generated prior to the athletic activity and/or do not substantially change during the athletic activity. The inputs may also include any of the real-time inputs discussed herein.

At operation 492, an adjusted initialization pacing target or target range is generated, by the pacing engine, based on the static inputs of the metabolic inputs, mechanical inputs, and/or environmental inputs received in operation 490. The direction (e.g., increasing the pace or reducing the pace) is based on the particular values of the inputs, as discussed above. In addition, the amount that the initialization pacing target or target range is adjusted may be based on the magnitude of the value of the inputs.

At operation 494, based on the adjusted initialization pacing target or target range generated in operation 492 and the real-time inputs of the metabolic inputs, mechanical inputs, and/or environmental inputs received in operation 490, a real-time pacing value is generated. The direction (e.g., increasing the pace or reducing the pace) is based on the particular values of the inputs, as discussed above. In addition, the amount that the real-time pacing value is adjusted may also be based on the magnitude of the value of the inputs. In some examples, the real-time pacing value is a particular target pace (e.g., a speed value) between the adjusted initialization target range or some difference from the adjusted initialization pacing target. In other examples, the real-time pacing value is an amount of speed or pace that the athlete should increase or decrease (e.g., a pacing delta value).

At operation 496, where the real-time pacing value includes a particular target pace, the real-time pacing value is compared to a current speed of the measurement platform that corresponds to the speed/pace of the athlete. The comparison is performed to determine if the target pace of the real-time pacing value is higher or lower than the current speed of the athlete and to determine the magnitude of that distance.

At operation 498, the measurement platform causes a physical output of the real-time pacing value. The output of the real-time pacing value may be based on the comparison performed in operation 496. For example, where the real-time pacing value is greater than the current speed of the measurement platform, a positive or speed-increase output is generated. Where the real-time pacing value is less than the current speed of the measurement platform, a negative or speed-decrease output is generated. As discussed above, the real-time pacing value may be a speed increase value or a speed decrease value. In such examples, a speed-increase output is generated for a speed-increase value of the real-time pacing value, and a speed-decrease output is generated for a speed-decrease value of the real-time pacing value. The speed-increase output may also include a magnitude based on how much the athlete should increase speed. The speed-decrease output may similarly include a magnitude based on how much the athlete should decrease speed.

The physical output for the real-time pacing value may be presented via audio elements (e.g., speakers), visual elements (e.g., display screens, single or multiple discrete lights), and/or haptics. The output elements or devices may be part of the measurement platform or may be part of a separate device that is in communication with the measurement platform. For instance, the output elements may be integrated into shoe or ankle mounted devices, arm bands, wrist devices, running glasses (or visual indicators connected to glasses), carbuds, etc. A first type of output may be generated for the speed-increase output, and a second type of output may be generated for the speed-decrease output. As an example, a first color (e.g., green) may be used for a speed-increase output and a second color (e.g., red) may be used for a speed-decrease output. Other visual indicators such as arrows, text, shapes, etc. may be used for the different types of output. The size or intensity of such shapes or objects may represent the magnitude of the speed-increase output or the speed-decrease output. A spectrum of colors (e.g., green-yellow-red) or symbols may also be displayed based on the speed-increase output and/or the speed-decrease output to indicate which direction to adjust pace and by how much. Similarly, for haptics or audio vibration or tone patterns may be generated to indicate a speed-increase output or a speed-decrease output. A continuous tone may also be generated in some examples where the pitch and/or volume of the tone changes based on real-time pacing value, such as whether there is a speed-increase output or a speed-decrease output and the magnitude of those outputs.

FIGS. 5A-C depict example plots of force curves for athletes that have different running styles and forms. When running, different athletes may strike the ground during a stride with different parts of their foot, which changes how the impact forces are experienced by the body of the athlete. For instance, some runners strike the ground with the forefoot section of their foot, some runners strike the ground with the midfoot section of the foot, and some runners strike the ground with the heel of their foot. The impact that is endured by the athlete over a run may affect how quickly the runner fatigues or becomes susceptible to injury. An analysis of the force curves generated from running impacts may be used to generate useful input data, such as impact loading rate and lower body stress, during a run that can be used as an input to the pacing engine to inform the protective pacing outputs.

FIG. 5A depicts a force curve plot 502 for an athlete that is a forefoot striker, FIG. 5B depicts a force curve plot 512 for an athlete that is a midfoot striker, and FIG. 5C depicts a force curve plot 522 for an athlete that is a heel striker. The vertical axis of the plots represents an impact force in units of body weight of the user. The impact forces may be vertical impact forces or a vertical ground reaction force. The horizontal axis of the plots represents a percentage of the ground contact time for the particular stride (e.g., how long the foot is in contact with the ground).

Each of the force curve plots 502, 512, 522 include an actual, directly measured force of impact over time (using a direct force sensor) and a modeled force of impact over time generated from the measurement platforms discussed herein. For instance, plot 502 includes an actual force curve 504 and a modeled force curve 506, plot 512 includes an actual force curve 514 and a modeled force curve 516, and plot 522 includes an actual force curve 524 and a modeled force curve 526. As can be seen from the plots 502, 512, 522, the modeled force curves 506, 516, 526 substantially match the actual force curves 504, 514, 516, which allows for use of the modeled force curves to be used in generating the impact metrics and related inputs to the pacing engine. For instance, a first fitting model may be generated for forefoot strikers, a second fitting model may be generated for midfoot strikers, and a third model may be generated for heel strikers. In some examples, one model may be used for both midfoot and forefoot strikers and a different model may be used for heel strikers.

As can be seen from the different plots 502, 512, 522, the force curves for different types of strikers may be different. For instance, the actual forefoot striker force curve 504 shape is substantially parabolic or Gaussian with a single peak. The actual midfoot striker force curve 514 is also substantially parabolic or Gaussian with a single peak. The initial ramp up of impact in the first 50% of ground impact time, however, differs. The actual heel striker force curve 524 shape is substantially different from the actual midfoot striker force curve 514 shape and the actual forefoot striker force curve 504 shape. For instance, the heel striker force curve 524 shape includes two peaks, with the second peak being higher and wider than the first peak. The heel striker force curve 524 shape is similar to two different parabolic or sinusoidal curves that are combined together. The different shapes of the respective force curves for the different types of strikers may be used to model force curves and generate real-time force curves for athletes as they are running, as discussed further below.

FIG. 5D depicts an example method 550 for generating the modeled force curves from accelerometer/gyroscope measurements from the measurement platform. At operation 552, kinematic data is acquired or received over a stride from the athlete. The kinematic data may include the accelerometer data and/or gyroscope data (e.g., IMU data or motion data), from an accelerometer and/or gyroscope of a measurement platform attached to the athlete. For instance, the accelerometer and/or gyroscope may be positioned in a foot pod attached to a shoe of the athlete. The time series of accelerometer data acquired in operation 552 may then be further processed and analyzed in method 550 as discussed further below. At operation 553, pressure sensor data may also be obtained over a stride for the athlete. For example, a pressure sensor that is incorporated into the foot pod or within the shoe or insole may measure pressure changes of the foot as the foot impacts the ground. Position and/or barometric data may also be received or acquired in operation 551 over a stride of the athlete. The position data may be acquired from a global positioning sensor, and the position data may include GNSS data or similar data. The barometric data may be acquired or received from the barometer of the measurement platform. The position data and/or barometric data may be used to determine incline and/or altitude. The position data may also be used to determine speed, velocity, and/or acceleration in some examples.

At operation 554, ground contact time or an initial ground contact is determined based on acceleration changes identified in the kinematic data and/or pressure changes identified in the pressure data. For instance, an initial impact signature may be identified when the force associated with the initial impact is detected (e.g., a steep increase in force), and a ground-release signature may be identified when the force (or conclusion of impact force) is detected from the foot leaving the ground. The time between such detection events is the ground contact time for a particular stride. In yet other examples, external imaging data of the athlete may be available, and the external imaging data may be utilized to determine the ground contact time. The external imaging data may come from a laser source, an infrared camera, or a visible-spectrum camera, among others. In still other examples, a laser system may be used to determine ground contact. For instance, if a laser beam near the ground is tripped or blocked by the foot, that data may be used to determine when the foot contacts the ground and for how long the foot contacts the ground.

At operation 556, a foot angle is determined from the kinematic data. In examples where the accelerometer/gyroscope is positioned within a foot pod attached to a shoe, the pitch or angle of the foot as it strikes the ground and contacts the ground during the ground contact time may be determined based on the pitch of the accelerometer/gyroscope and/or velocities/speeds determined from the kinematic data. Accordingly, based on a foot angle, an identification may be made as to whether the athlete is a heel striker, midfoot striker, or forefoot striker.

At operation 558, based on the foot angle determined in operation 556, a fitting model is selected. For instance, if the foot angle indicates that the athlete is a heel striker, a heel-striking fitting model may be selected. Similarly, if the foot angle indicates that the athlete is a forefoot striker, the forefoot-striker model may be selected.

At operation 560, the kinematic data is fitted to the fitting model (that was selected in operation 558) to generate a modeled force curve, such as the modeled force curves 506, 516, 526. Additional assumptions or metrics may be integrated into the particular model or used when fitting the data to the fitting model. For instance, the total bodyweight (e.g., area under the force curve) should be equal to one total bodyweight for the athlete over the ground contact time. The force while the foot is on the ground plus the force while the foot is in the air over a stride must equal one bodyweight, and the force while the foot is in the air is equal to zero. This understanding allows for a determination of how much area under the curve is required and the ground contact time provides a start and end point. The remainder of fitting the force curve is defining the shape of the force profile, which is provided by the selected fitting model.

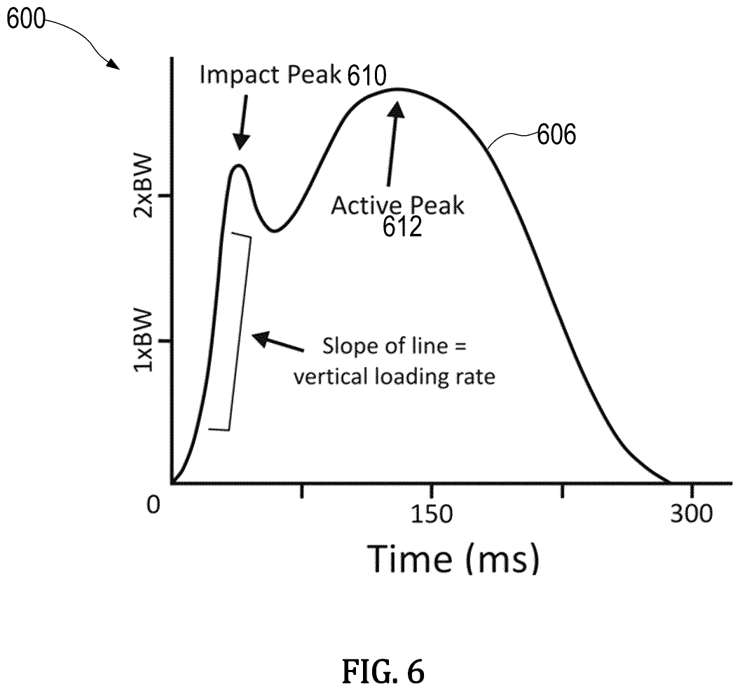

At operation 562, based on the modeled force curve generated in operation 560, one or more impact metrics, such as impact loading rate, may be determined. One example of generating impact metrics is discussed below with reference to FIG. 6. At operation 564, a lower body stress value is generated by accumulating or summing one or more of the impact metrics generated in operation 562. For example, the impact loading rate for each stride may be accumulated over time, such as over the athletic activity being performed or over a plurality of athletic activities being performed over a time frame (e.g., set number of days, a week, etc.). As one example, a total lower body stress score may be generated for each week of athletic activities performed by the athlete. Thus, the lower body stress experienced by the athlete may be tracked over time.

Impact metrics may also be generated in other manners outside of the use of the force curves discussed above. For instance, as shown in FIG. 5, method 550 may include alternatively or additionally performing operations 570 or 572 to then generate the impact metrics at operation 562. For example, when operation 570 is implemented, operation 554 may be include determining the initial contact of the foot with the ground. As discussed above, the acceleration change or pressure change when the foot impacts the ground is significant. That time at which the significant acceleration change is identified may be considered the initial impact time.

At operation 570, the acceleration or pressure is integrated over an initial impact period representing the impact forces experienced for the stride. In some examples, only the vertical component of acceleration may be integrated. The initial impact period may be a set amount of time, such as between 20-50 ms or 30-100 ms. In other examples, the initial impact period may be variable and based on the changes in acceleration. For instance, the initial impact period may be between the initial ground contact and a detected peak in the acceleration. In other examples, the initial impact time may be a percentage of the ground contact time, such as the first 10%, 20%, 30%, 40%, or 50%. At operation 562, impact metrics may be generated based on the integrated acceleration in the initial impact period. In some examples, the impact metrics may be the value of such integrated acceleration. In other examples, the impact metrics may be based on a height of the peak acceleration or pressure (e.g., peak amplitude) and/or the position of the peak amplitude during the ground contact time (e.g., at what percent duration of the ground contact time did the peak impact occur). At operation 564, those impact metrics may be accumulated over the athletic activity and/or over multiple activities to determine a lower body stress value.

Operation 572 may be similar to operation 570. For instance, in operation 572 speed and incline are determined at the initial impact time determined in operation 562. The speed and/or incline may be determined from the position data, barometric data, and/or the kinematic data. The speed and incline may then be used to estimate impact metrics. The impact metrics may be relative metric that is based on the combination of the speed and incline. For example, higher speeds generally lead to higher impact forces. Running downhill may also lead to higher impact forces than running uphill. At operation 564, those impact metrics may be accumulated over the athletic activity and/or over multiple activities to determine a lower body stress value. In yet other examples, the overall speed and incline for the runner may be tracked throughout the athletic activity without being directly tied to the initial impact time. The impact metrics may then be generated based on the overall speed and incline for the run, and the impact metrics may be aggregated to generate a lower body stress value.

As another option, a regression or ML model may be used to generate the impact metrics. For example, at operation 574, a plurality of activity metrics may be generated from the accelerometer and/or gyroscope data. The activity metrics may include any of the metrics discussed herein, such as speed, incline, foot angle, and ground contact time, among others. At operation 576, the activity metrics are provided as input into a trained ML model, regression model, or similar model. The model processes the activity metrics, and the output of the model is used to generate the impact metrics at operation 562. In some examples, the output of the model includes the impact metrics. In other examples, the output of the model is further processed or converted to generate the impact metrics.

FIG. 6 depicts an example annotated force curve plot 600 with a modeled force curve 606 for a stride of an athlete that is a heel striker. The force curve 606 includes a first maxima that corresponds to an impact peak 610. The force curve 606 also includes a second maxima that corresponds to an active peak 612 that occurs about halfway through the ground contact time of the stride and corresponds to the push off force from the athlete when the athlete pushes off from the ground. The slope of the force curve 606 from the start of the ground contact time to the first impact peak 610 is the impact or vertical loading rate. The slope may be the average change between the start point (e.g., zero force at zero time) and the impact peak 610. In other examples, the slope may be calculated from the initial or start time to different specified point on the force curve 606, such as when the force curve is at a set percentage of ground contact time (e.g., 10%, 15%, 20% of ground contact time). In still other examples, the slope may be calculated from the initial or start time to a point on the force curve 606 where the force curve reaches a percentage of body weight, such as 75%, 100%, 125%, 150% of body weight.

In addition to receiving impact loads on the body when the athlete strikes the ground, the athlete also experiences negative mechanical power when the foot strikes ground due to the frictional resistance of the surface of the ground. For example, while the athlete's foot is on the ground during a stride, the ground resists the movement of the foot relating in negative mechanical power. The athlete must the increase the amount of the power exerted to overcome this negative mechanical power to continue running at the same pace or to increase speed.

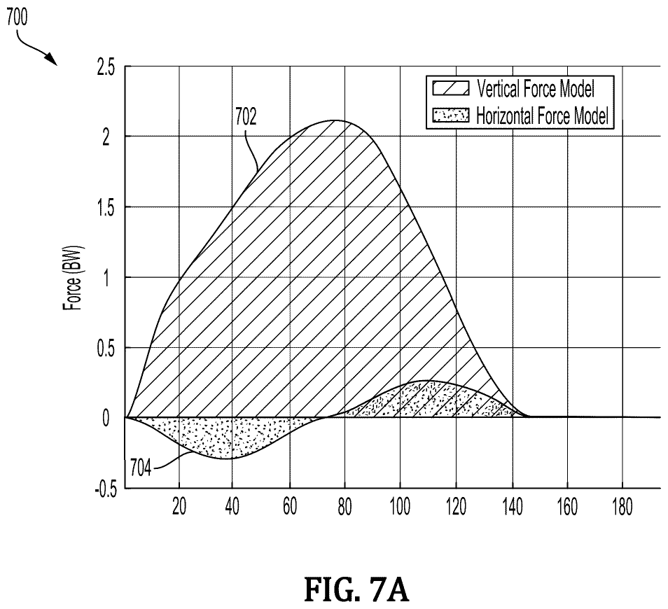

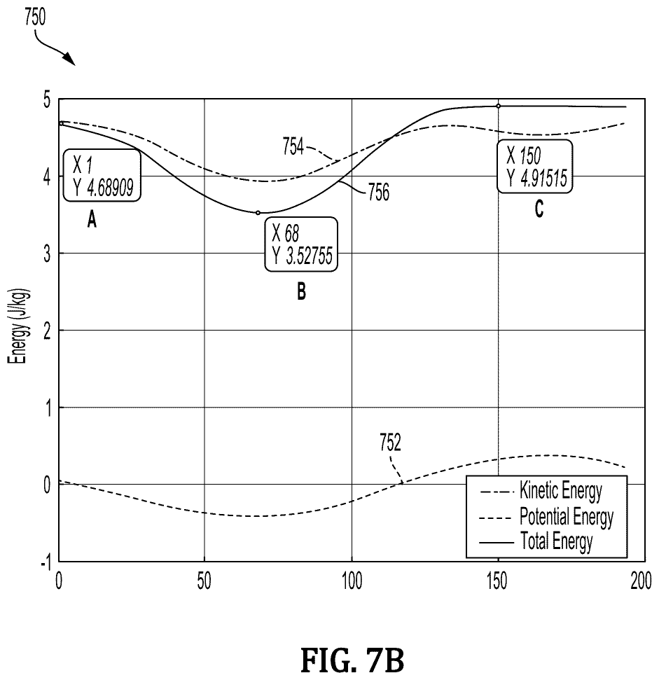

FIG. 7A depicts an example force plot 700 that depicts a modeled vertical force curve 702 and a modeled horizontal force curve 704. The plot 700 has a vertical axis representing force and the horizontal axis represents time. The modeled vertical force curve 702 may be generated as discussed above. The modeled horizontal force curve 704 may be generated as discussed further below. For instance, the horizontal force curve measuring a speed change from a current stride to the next stride, and the changes in speed result in changing horizontal force curves. The current incline may also be factored into generating the horizontal force curves. Additional factors that may be used in generating the horizontal force curves include back kick height, ground time, speed of the runner, peak foot speed during a swing phase, toe-off foot velocity, the foot strike type of the runner (e.g., heel striker, midfoot striker, or forefoot striker), and/or the foot angle through the ground time.

As can be seen from the plot 700, the horizontal force curve 704 includes a negative component, in the first portion of the ground contact time of the stride, and a positive component in the second portion of the ground contact time of the stride. This change in force is due to how the runner interacts with the ground during a stride. For instance, the runner slows when touching the ground initially and then pushes off and speeds back up. The kinetic energy and mechanical potential energy of the runner also changes throughout the stride.

FIG. 7B depicts an example energy plot 750 based on the force plot 700 of FIG. 7A. For instance, the energy plot 750 corresponds to the same stride of the athlete that is represented in the force plot 700 of FIG. 7A. The energy plot 750 includes a kinetic energy curve 754 that represents the kinetic energy of the center of mass of the athlete, a potential energy curve 752 that represents the mechanical potential energy of the athlete, and a total energy curve 756 that is the sum of the kinetic energy curve 754 and the potential energy curve 756.

Calculation of the energy curves in the plot 750 may be based on the modeled force curves in the force plot 700. For instance, the kinetic energy may be determined according to the following equation:

$$KE = \text{.5} m * {v}^{2} , \qquad ( Eqn . 1 )$$

where KE is the kinetic energy, m is the mass of the athlete, and v is the velocity of the athlete. The velocity may be calculated based on the integrating the acceleration of the athlete, which may in turn be based on the force divided by the athlete's mass. The energy may be based on both the vertical and horizontal force curves. The integration of the acceleration results in a change in velocity and the current or absolute velocity may be determined from motion data of the previous step to get the runner's speed at the moment of touch down with the ground. Accordingly, based on the known (or received) mass of the user and the modeled horizontal force curve 704 and/or vertical force curve 702, the kinetic energy curve 754 may be generated.

The potential energy may be calculated according to the following equation:

$$P E = m * g * h , \qquad ( Eqn . 2 )$$

where m is the athlete's mass, g is gravity, and h is height which can be determined by integrating the vertical component of velocity described above.

The total energy curve 752 may then be generated by summing the kinetic energy curve 754 and the kinetic energy curve. As discussed above, during a stride, the runner slows when touching the ground initially, then pushes off and speeds back up. As a result, the runner's total energy begins at a value before impact point (A), then decreases to a minimum point (B), and then increases again when pushing off back into the air (C).

Negative mechanical power for the particular stride may be calculated by determining the difference between the total energy at points A and B over the time interval between points A and B that represents the impact absorption phase of the stride. Positive mechanical power for the stride may be calculated by determining the difference between the points B and C over the time interval between points B and C that represents the push off portion of the stride. On flat ground, to achieve the same speed, the positive mechanical power and the negative mechanical power are equal to one another. On flat ground, if the positive mechanical power is greater than the negative mechanical power, the athlete will increase in speed.

As discussed above, the equipment of the athlete may also make a difference in both the performance and the safety of the runner. For instance, the configuration or composition of a runner's shoe affects how efficiently and safely a runner may be able to run. One measure or property of a shoe is an energy return measure for the shoe.

FIG. 8 depicts an example energy return plot 800. The energy return plot includes a vertical axis representing force and a horizontal plot representing deformation of the sole of the shoe over a stride. The plot 800 includes a compression curve 802 that occurs when the shoe is absorbing impact of the stride. The plot 800 also includes a decompression curve 804 that occurs when the sole of the shoe is decompressing after the compression due to impact. The energy that is lost during the stride due to compression of the shoe is represented by the area between the compression curve 802 and the decompression curve 804. The energy return from the shoe is represented by the area under the decompression curve 804. In the example depicted, the energy return of the particular shoe is about 75%.

Determining energy return for a shoe has generally required a laboratory environment with specialized equipment (e.g., a single dimensional plunger) that is used to generate the compression curve 802 and the decompression curve 804. With the measurement platforms of the present technology, the energy return of the shoe may be generated without the need for such specialized equipment and may be generated from data generated while an athlete is actually performing an athletic activity on a real trail or other types of ground surfaces.

As an example, from the force curves discussed above and a position determination from the measurement platform, the relative energy return for a particular shoe may be determined. For instance, the forces experienced may be determined from the vertical force curve, and a center of pressure may also be determined from the accelerometer/gyroscope data. The center of pressure indicates a position of the sole of the shoe. The center of pressure is highly correlated with the foot angle when the foot is in contact with the ground. The foot angle may be measured by the foot pod of the present technology. By having the foot angle of the foot at impact and through the toe-off in all three dimensions, the center of pressure can be modeled. The foot impact and toe-off can be determined with high accuracy based on the acceleration measurements and the angular features, which also allows for a determination of where the center of pressure begins and ends along with the course it takes throughout the contact phase.

For a hypothetical shoe with no compression (e.g., a perfectly rigid sole), a motion pattern or model can be generated for how the center of pressure for that shoe should move throughout the ground contact time of the stride. Different patterns or models may be generated for the different types of foot strikers as well (e.g., heel-striker, midfoot-striker, forefoot striker).

During the actual athletic activity, the real-time center of pressure may be tracked and compared to the perfectly rigid model. The amount and/or type of deviation of the real-time center of pressure from the perfectly rigid model indicates the amount of compression that the shoe experiences at each point in the ground contact time, which can be correlated with the amount of vertical force at the same point in the ground contact time. The forces and velocities recorded, along with the determined compression, may then be used to determine or estimate the energy return of the shoe. The amount of deformation that is detected or determined by the measurement platform may also be representative of how much cushion is provided by the shoe. Accordingly, the cushion data and/or energy return data used as input for the pacing engine may be based on or determined from the compression data generated by the measurement platform.

FIG. 9 depicts an example energy return plot 900 for a sample of different shoes. The vertical axis of the plot 900 represents the energy return estimated or calculated based on measurements from the measurement platform, and the horizontal axis of the plot 900 represents different shoes. In the example plot, three data points are provided representing three different shoes. A first data point 902 represents the energy return for a first shoe, a second data point 904 represents the energy return for a second shoe, and the third data point 906 represents the energy return for a third shoe. The energy return estimate on the vertical axis is relative to energy return of Shoe 3. For instance, the energy return of Shoe 1 was roughly 85% of the energy return of Shoe 3. The energy return of Shoe 2 was roughly 110% of the energy return of Shoe 3.

The first shoe has the lowest energy return, the second shoe has the highest energy return, and the third shoe has an energy return that is between the first shoe and the second shoe. Accordingly, a runner wearing the second shoe may be able to run at a faster pace without exerting as much additional energy as compared to a runner wearing the first shoe.

As discussed above, environmental inputs also affect the protective pacing feedback. As an example, the current incline of an athlete affects how fast that athlete should run. For instance, pacing for running uphill versus uphill is generally different. The current incline for an athlete may be determined in a multitude of manners. For instance, an instantaneous derivative of altitude can be approximated by combining timed barometric pressure samples and pace data to allow fitting of a linear incline function to determine instantaneous incline. Incline can also be determined by pressure measurements (using a pressure sensor, such as a barometer, pressure altimeter, and/or the like) over time. For example, a pressure sensor can be used to detect changes in a user's elevation (e.g., attitude or vertical position). Alternatively or in addition, incline can be estimated using the position and direction estimation techniques described herein and a map, stored on the measurement platform, translating from position and direction to incline. An example of such a map and generation of the map is discussed below in FIGS. 10-11.

FIG. 10 depicts an example topographic aggregate trail map 1000. The trail map 1000 is generated from the aggregate position and altitude data from a plurality of athletes running the trail with the measurement platform of the technology discussed herein. While some trail maps already exist that are created by cartographers or others that draw maps on top of landscapes, such preexisting maps lack the resolution of data to determine a particular incline of the athlete at all points on the map. For instance, the altitude for substantially each step along the trail is not available. Accordingly, to address this problem and generate a trail map with higher resolution altitude data along the trail, the aggregate trail map 1000 may be generated from the aggregate data of multiple athletes. The trail map may be defined by a trail or route marker 1002 and the trail data may be defined at each position by (1) a latitude value, (2) a longitude value, and (3) an altitude and/or incline value, where the trail data is generated from an aggregate collection of data of athletes. The incline value may be captured based on the accelerometer data and/or the barometer (e.g., pressure) data collected from the measurement platform of each of the athletes in the aggregate data set as the athletes run along the trail. As a result, when an athlete is at a particular location on the aggregate trail map 1000, such as a location 1004, the altitude and/or incline value may be accessed from the trail data and used as an incline input as discussed herein.

FIG. 11 depicts an example method 1100 for generating and accessing a topographic aggregate trail map. At operation 1102, global positioning data (e.g., GNSS data) is acquired from the multiple measurement platforms that are worn by multiple athletes running the same trail. The global positioning data may include a longitude position, a latitude position, and a time stamp. At operation 1104, accelerometer and/or pressure data (e.g., generated from barometers) is acquired from the multiple measurement platforms. The accelerometer and/or pressure data is acquired during the same runs for which the global positioning data was acquired. The accelerometer and/or pressure data may also include time stamps.

At operation 1106, accelerometer and/or pressure data may be correlated with the global positioning data from each of the measurement platforms. For instance, for a particular measurement platform, the global positioning data may be correlated with the accelerometer and/or pressure data by matching the time stamps of the global positioning data and the time stamps of the accelerometer and/or pressure data. Accordingly, for each measurement platform, a time series of arrays or tuples of data may be generated from the correlated global positioning data and the accelerometer and/or pressure data. An example array of data at a single point in time may be {time, latitude, longitude, accelerometer data, pressure data}. Other examples may include additional or fewer elements in the array.

At operation 1108, the correlated global positioning data and accelerometer and/or pressure data is combined to form an aggregate data set for the trail. The combining or aggregation of the correlated data may include an averaging of the accelerometer data and/or pressure data for each of the latitude and longitude positions along the trail, resulting in high-resolution data for the trail. For instance, aggregate data may be available for resolutions of less than a foot. The pressure data may also be converted to altitude data during or prior to the combination operation such that an altitude is available for each longitude and latitude position along the trail.

At operation 1110, incline data for the global positions of the trail is generated from the aggregated data. For instance, where altitude data is used, the incline may be determined from the altitudes of adjacent global positions along the trail. Where accelerometer data is used, the incline data may be based on the incline data determined for each of the measurement platforms during the respective runs by combining timed barometric pressure samples and pace data to allow fitting of a linear incline function to determine instantaneous incline. The incline data may then be paired with the particular geolocation data to form arrays having incline data for each point on the trail. For instance, the array may be {latitude, longitude, incline}.

The incline value may also be associated with a directionality. For instance, if the runner is running the trail in a first direction (e.g., uphill direction) the incline value is positive, and if the runner is running the trail in the opposite direction (e.g., the downhill direction) the incline value is negative. However, the absolute value of the incline may not change as the actual geographical features of the trail do not change.

At operation 1112, the aggregate trail map is generated that includes incline data generated at operation 1110 for the latitude and longitude positions along the trail. In some examples, the aggregate trail map may not include the incline data but may include the altitude data for each of the latitude and longitude positions. In yet other examples, the aggregate trail map may include both the altitude data and the incline data.

Once the aggregate trail has been generated, the aggregate trail map may be stored in a remote storage location, such as on a server or an Internet-accessible database that is accessible by the measurement platforms of athletes. Accordingly, while an athlete is running, the measurement platform of the athlete may be accessed. In some examples, the aggregate trail map may be accessed in real time for the remote storage location via an Internet connection. In other examples, the aggregate trail map may be downloaded locally to the measurement platform and accessed locally during the run.

At operation 1114, after the aggregate trail map has been generated, the aggregate trail is accessed, and incline data is generated from the aggregate trail map. In examples where the aggregate trail map directly includes incline data (such as the incline data generated in operation 1110), a direct lookup of incline data may be performed on the aggregate trail map. For instance, as global positioning data is received by the measurement platform during the run, the global positioning data is used to query the aggregate trail map to receive incline data for the particular position. The direction of the runner may also be determined from consecutive time series of the global positioning data, and the determined direction of the runner may be used to alter the sign (e.g., positive or negative) of the incline data.

In examples where the aggregate trail map includes altitude data for each of the latitude and longitude pairs, the incline data may be generated by determining the slope between altitudes associated with the two consecutive latitude and longitude pairs received by the measurement platform. For instance, for a first latitude and longitude pair indicated by global positioning data received at first time, a first altitude is received. For a second latitude and longitude pair indicated by global positioning data received at a second time that is subsequent the first time, a second altitude is received. The difference between the first altitude and the second altitude divided by the distance between the first and second latitude/longitude pairs indicates the incline, and that incline data may be used for generating a real-time pacing output as discussed herein.

At operation 1116, the aggregate trail map may be updated based on sensor data acquired by the measurement platform that accessed the aggregate trail map in operation 1114. For example, as the measurement platform is moving along the trail, the measurement platform also may generate pressure data along with the acquired GNSS data. That pressure data may be used to update or further refine the aggregate trail map. As a result, the aggregate trail map may continue to be refined as more measurement platforms are used and as more runs of the trail are taken. In addition, as the trail changes, such as through erosion, the aggregate trail map continues to update and track the trail changes through the continued updates from the data collected by het measurement platforms. Such a continuously updating trail map also reduces or eliminates the need for the use of traditional survey or other cartography tools.

Aspects of the present invention, for example, are described above with reference to block diagrams and/or operational illustrations of methods, systems, and computer program products according to aspects of the invention. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved. In some examples, not all operations may be performed. Further, as used herein and in the claims, the phrase “at least one of element A, element B, or element C” is intended to convey any of: element A, element B, element C, elements A and B, elements A and C, elements B and C, and elements A, B, and C.

The description and illustration of one or more examples provided in this application are not intended to limit or restrict the scope of the invention as claimed in any way. The aspects, examples, and details provided in this application are considered sufficient to convey possession and enable others to make and use the best mode of claimed invention. The claimed invention should not be construed as being limited to any aspect, example, or detail provided in this application. Regardless of whether shown and described in combination or separately, the various features (both structural and methodological) are intended to be selectively included or omitted to produce an example with a particular set of features. Having been provided with the description and illustration of the present application, one skilled in the art may envision variations, modifications, and alternate examples falling within the spirit of the broader aspects of the general inventive concept embodied in this application that do not depart from the broader scope of the claimed invention.

We claim:

1. A measurement system that is retained by an athlete during an athletic activity, the measurement system comprising:
a housing that includes:
an accelerometer;
a gyroscope;
at least one processor; and
at least one memory, the at least one memory storing instructions that, when executed by the at least one processor cause the system to perform operations comprising:
receiving accelerometer data and gyroscope data generated by the accelerometer and the gyroscope during a ground-contact portion of a stride;
generating one or more impact metrics based on at least one of the accelerometer data or the gyroscope data;
generating a real-time pacing value based on the generated one or more impact metrics; and
causing a physical output based on the generated real-time pacing value.

2. The measurement system of claim 1, wherein the operations further comprise:
determining an initial impact time for the stride;
integrating a vertical acceleration component of the acceleration data over an initial impact period; and
wherein generating the one or more impact metrics is based on the integration of the vertical acceleration component.

3. The measurement system of claim 1, wherein the operations further comprise:
determining speed and incline at an impact with the ground during the stride; and
wherein generating the one or more impact metrics is based on speed and incline.

4. The measurement system of claim 1, wherein causing the physical output includes generating at least one of an audio output, a visual output, or a haptic output.

5. The measurement system of claim 1, wherein the operations further comprise:
based on the accelerometer data and gyroscope data, generating a modeled force curve for the ground-contact portion of the stride; and
based on the modeled force curve, determining an impact loading rate for the stride, wherein the impact loading rate is one of the one or more impact metrics.

6. The measurement system of claim 5, wherein generating the impact loading rate includes determining a slope of the modeled force curve.

7. The measurement system of claim 6, wherein the slope of the modeled force curve occurs in a section of the force curve between a starting point of the force curve and a first peak of the force curve.

8. The measurement system of claim 1, wherein the operations further comprise:
calculating the impact loading rates for a plurality of strides during the athletic activity; and
generating a lower body stress value by accumulating the calculated impact loading rates.

9. The measurement system of claim 8, wherein the athletic activity is a first athletic activity and the operations further comprise, during a second athletic activity generating a second pacing value based on the lower body stress value.

10. The measurement system of claim 1, wherein the housing is a foot pod that is configured to attach to a shoe during the athletic activity.

11. A measurement system that is retained by an athlete during an athletic activity, the measurement system comprising:
one or more housings that house:
an inertial measurement unit including an accelerometer and a gyroscope;
at least one environmental sensor;
at least one processor; and
at least one memory, the at least one memory storing instructions that, when executed by the at least one processor cause the system to perform operations comprising:
based on accelerometer and gyroscope data generated by the accelerometer and the gyroscope, generating one or more real-time mechanical inputs;
based on environmental data generated by the at least one environmental sensor, generating one or more real-time environmental inputs;
accessing at least one of a metabolic static input or an equipment static input;
based on the at least one of a metabolic static input or an equipment static input, adjusting an initialization pacing target to form an adjusted initialization pacing target;
based on the one or more real-time environmental inputs and the one or more real-time mechanical inputs, generating a real-time pacing value; and
causing a physical output based on the generated real-time pacing value.

12. The measurement system of claim 11, wherein:
the initialization pacing target is a target range having a maximum pacing threshold and a minimum pacing threshold;
the adjusted initialization pacing target includes an adjusted maximum pacing threshold and an adjusted minimum pacing threshold; and
the real-time pacing value is between the adjusted maximum pacing threshold and the adjusted minimum pacing threshold.

13. The measurement system of claim 11, wherein the metabolic static input includes at least one of recovery, sleep, or nutrition.

14. The measurement system of claim 11, wherein the equipment input includes at least one of apparel data or shoe data.

15. The measurement system of claim 11, wherein the one or more real-time mechanical inputs include at least one of impact loading rate, negative mechanical power, or positive mechanical power.

16. The measurement system of claim 11, wherein the one or more real-time environmental inputs include ground surface data.

17. The measurement system of claim 11, further comprising a global positioning sensor, wherein the one or more real-time environmental inputs include incline data, wherein the incline data is generated from querying an aggregate trail map based with global navigation satellite system (GNSS) data generated from the global positioning sensor during the athletic activity.

18. A measurement system that is retained by an athlete during an athletic activity, the measurement system comprising:
one or more housings that house:
an inertial measurement unit including an accelerometer and a gyroscope;
a barometer;
a temperature sensor;
at least one processor; and
at least one memory, the at least one memory storing instructions that, when executed by the at least one processor cause the system to perform operations comprising:
determining an altitude based on at least one of measurements from the barometer or position data received from a global positioning sensor;
measuring, by the temperature sensor, a temperature of an environment around the measurement system;
generating a real-time pacing value based on the altitude and temperature; and
causing a physical output based on the generated real-time pacing value.

19. The measurement system of claim 18, wherein an increase in altitude causes a reduction in the real-time pacing value.

20. The measurement system of claim 18, wherein an increase in temperature causes a reduction in the real-time pacing value.
